(19) United States
(12) Patent Application Publication
(10) Pub. No.: US 2024/0050356 A1
(43) Pub. Date: Feb. 15, 2024

(54) ANTIOXIDANT COMPOSITIONS

(71) Applicant: INTERNATIONAL FLAVORS & FRAGRANCES INC., New York, NY (US)

(72) Inventors: Manon Gilles, Clichy (FR); Stéphanie Woimant, Conflans Sainte Honorine (FR); Frederique Carpuat, La Garenne Colombes (FR)

(21) Appl. No.: 18/255,652

(22) PCT Filed: Dec. 7, 2021
(86) PCT No.: PCT/US2021/062087

§ 371 Date: Jun. 2, 2023

Related U.S. Application Data
(60) Provisional application No. 63/122,221, filed on Dec. 7, 2020.

(30) Foreign Application Priority Data
Dec. 18, 2020 (EP) 20306614.7

(51) Int. Cl. A61K 8/67; A61K 8/37; A61K 8/49; A61K 8/365; A61K 8/362; A61Q 13/00; A61Q 5/02; A61Q 5/12

(52) U.S. Cl. CPC A61K 8/678; A61K 8/37; A61K 8/4926; A61K 8/365; A61K 8/362; A61Q 13/00; A61Q 5/02; A61Q 5/12; A61K 2800/522

(57) ABSTRACT

Disclosed are antioxidant compositions containing a primary antioxidant, an organic acid, and optionally tris(tetramethylhydroxypiperidinol) citrate. Also disclosed are fragrance compositions or consumer products containing such an antioxidant composition.

### CROSS REFERENCE OF RELATED APPLICATIONS

This application is a 371 of International Application No. PCT/US2021/062087, filed 5 Dec. 7, 2021, which claims the benefit of 63/122,221, filed Dec. 7, 2020, which are incorporated by reference in their entirety.

### FIELD OF THE INVENTION

This invention relates to antioxidant compositions for use in fragrances, cosmetics, and consumer products as alternatives to butylated hydroxytoluene (BHT).

### BACKGROUND OF THE INVENTION

Fragrances are widely used in consumer products including personal care, home care and fabric care products.

Generally, a fragrance composition contains natural and synthetic ingredients such as terpenes (e.g., limonene and α-terpinene), alcohols (e.g., citronellol), phenols (e.g., eugenol), aldehydes (e.g., triplal), ketones (e.g., γ-methyl ionone), esters (e.g., methyl dihydro jasmonate), lactones (e.g., nonalactone-γ), and ethers (e.g., cedramber). Many are prone to oxidative degradation leading to the loss of olfactory character, the appearance of off-notes, undesirable color modifications, or the formation of irritant or allergenic substances. Antioxidant compositions are added to fragrances, cosmetics, and consumer products to inhibit oxidation.

Butylated hydroxytoluene (BHT) is widely used as an antioxidant because of its great efficiency and low cost. However, BHT can induce allergic reactions in the skin and other health issues. European Chemicals Agency (ECHA) has already restricted its use due to possible endocrine disrupting properties. α-Tocopherol, the most biologically active form of vitamin E molecules, is used as a skin care active in cosmetic products at a relatively high concentration due to its antioxidant property. Nevertheless, it is undesirable to use a high level of α-tocopherol in fragrances or cosmetics as an antioxidant, not only because it is costly, but also due to the bad smell after it is oxidized. Consumers prefer antioxidant compositions alternative to BHT from a sustainable and cost-effective source.

To avoid fragrance oxidative degradation, new antioxidant alternatives should be found either by identifying the most efficient and safe synthetic or natural antioxidant molecules or by taking benefits from the synergies between antioxidants and other ingredients in the formula.

There is a need to develop antioxidant compositions having low or no odor, color and evolutions overtime. They should be widely soluble in different media such as water, ethanol and non-polar media (corresponding to essential oil media). Further, those antioxidant compositions should be safe without the tendency of forming irritant or allergenic substances.

### SUMMARY

The present invention has been accomplished by the surprising discovery of certain antioxidant compositions comprising a primary antioxidant and an organic acid. Exemplary primary antioxidants are α-tocopherol, methyl 3-(3.5-di-tert-butyl-4-hydroxyphenyl) propanoate, and a combination thereof. The organic acid can be selected from the group consisting of citric acid, oxalic acid, malic acid, and combinations thereof. Preferably, some antioxidant compositions contain tris(tetramethylhydroxypiperidinol) citrate (CAS No. 220410-74-2; “TTMHPC”). In addition, some antioxidant compositions contain a polar or non-polar solvent, or a combination thereof, including benzyl benzoate, triethyl citrate, and dipropylene glycol.

Antioxidant synergy was unexpectedly observed between the primary antioxidant and the organic acid, in the presence and absence of tris(tetramethylhydroxypiperidinol) citrate. The weight ratio of the primary antioxidant and the organic acid is preferably 500:1 to 1:50, more preferably 300:1 to 1:20, and most preferably 200:1 to 1:10.

Also within the scope of this invention are fragrance compositions or consumer products comprising any of the antioxidant compositions described above. In these fragrance compositions or consumer products, the primary antioxidant is present at a level of 0.001 wt % to 1 wt %, preferably 0.01 wt % to 0.3 wt %, and more preferably 0.01 wt % to 0.2 wt %; the organic acid is present at a level of 0.00001 wt % to 2 wt %, preferably 0.0001 wt % to 1 wt %, and more preferably 0.0001 wt % to 0.5 wt %; and tris(tetramethylhydroxypiperidinol) citrate is present at a level of 0 to 1 wt % (w/w), preferably 0.00001 wt % to 0.5 wt %, and more preferably 0.0001 wt % to 0.1 wt %.

As used herein, the term “primary antioxidant” refers to free a radical scavenger that reacts with chain-propagating radicals such as peroxyl, alkoxy, and hydroxy radicals in a chain terminating reaction. Specifically, these antioxidants donate hydrogen to the alkoxy and hydroxy radicals which transfer them into inert alcohols and water respectively. Typical commercial primary antioxidants are hindered phenols and secondary aromatic amines. The most widely used primary antioxidants are sterically hindered phenols. They are very effective radical scavengers during both processing and long-term thermal ageing and are generally olfactively inert and non-discoloring.

All parts, percentages and proportions refer to herein and in the claims are by weight unless otherwise indicated.

The values and dimensions disclosed herein are not to be understood as being strictly limited to the exact numerical values recited. Instead, unless otherwise specified, each such value is intended to mean both the recited value and a functionally equivalent range surrounding that value. For example, a value disclosed as “50%” is intended to mean “about 50%.”

The terms “L”, “mL”, and “μL” refer to “liter”, “milliliter”, and “microliter”, respectively.

As used herein, the singular forms “a,” “an,” and “the” include plural references unless the context clearly dictates otherwise. Unless defined otherwise, all technical and scientific terms used herein have the same meanings as commonly understood by one of ordinary skill in the art. When used in this document, the term “comprising” (or “comprises”) means “including (or includes), but not limited to.” When used in this document, the term “exemplary” is intended to mean “by way of example” and is not intended to indicate that a particular exemplary item is preferred or required. In this document, when terms such “first” and “second” are used to modify a noun, such use is simply intended to distinguish one item from another; and is not intended to require a sequential order unless specifically stated.

The details of one or more embodiments of the invention are set forth in the description below. Other features, objects, and advantages will be apparent from the description and the claims.

### DETAILED DESCRIPTION

It has been found that organic acids and optionally tris(tetramethylhydroxypiperidinol) citrate unexpectedly enhance the antioxidant capacity of a primary antioxidant, making their mixture very useful in the protection of fragrance compositions, fragrance ingredients and consumer products against oxidative degradations overtime.

The antioxidant compositions of this invention have shown improved preservation of fragrance compositions and consumer products either in polar or non-polar solvent and subjected to an oxidative stress. They provide a comparable protection against oxidation in a manner better than or similar to butylated hydroxytoluene (BHT), a traditional and less-desirable antioxidant.

Suitable primary antioxidants include α-tocopherol, methyl 3-(3,5-di-tert-butyl-4-hydroxyphenyl) propanoate (“MDTBHPP”), and a combination thereof. The term “α-tocopherol” include all its isomers including (±) α-tocopherol (CAS No. 10191-41-0), (+)-α-tocopherol (i.e., (2R)-2,5,7,8-Tetramethyl-2-[(4R,8R)-(4,8,12-trimethyltridecyl)]chroman-6-ol; CAS No. 59-02-9), and (−)-α-tocopherol (i.e., (25)-2,5,7,8-tetramethyl-2-[(4S,8S)-4,8,12-trimethyltridecyl]-3,4-dihydrochromen-6-ol; CAS Nos. 1406-18-4 and 77171-97-2). The preferred organic acid is citric acid.

Solvents are generally classified by their polarity, and considered either polar or non-polar, as indicated by the dielectric constant. However, it has to be noticed that polarity is a continuous scale and no strict boundary can be applied. Nonetheless, generally, solvents with dielectric constants greater than about 5 are considered “polar” and those with dielectric constants less than 5 are considered “non-polar.”

Various alternatives, modifications, variations or improvements may be made by those skilled in the art, each of which is also intended to be encompassed by the disclosed embodiments. The antioxidant systems disclosed herein are applicable to protect any fragrance raw materials (either natural or synthetic) against oxidation, such as 2,4-dimethyl-3-cyclohexene-2,5-carboxaldehyde, 2,6-dimethyloct-7-en-2-ol, (3-oxo-2-pentylcyclopentyl)acetic acid methyl ester, 1-(1,2,3,4,5,6,7,8-octahydro-2,3,8,8,-tetramethyl-2-naphthyl)ethan-1-one, 2,4-dimethylcyclohex-3-ene-1-carbaldehyde, 4-hydroxyl-3-methoxybenzaldehyde, trimethyl-2,6,6-bicyclo(3,1,1)hept-2-ene, 2-methoxy-4-(2-prop enyl)-phenol, (3aR,5aS,9aS,9bR)3a,6,6,9a-tetramethyl-2,4,5,5a,7,8,9, 9b-octahydro-1H-benzo[e][1]benzofuran, 3,7-dimethylocta1,6-dien-3-ol, methyl 2,4-dihydroxy-3,6-dimethylbenzoate, benzyl acetate, 4-(4-hydroxyphenyl)-butan-2-one, 5-hexyloxolan-2-one, 2-ethyl-3-hydroxy-4-pyranone, 3,7-dimethylocta-1,6-dien-3-yle acetate, 2H-1-benzopyrane-2-one, patchouli oil, bergamot oil, oud oil, cedarwood oil, Jasmin absolute, and the like.

The antioxidant composition described above can be applicable to protect any commercial products containing fragrances or fragrance raw materials, such as an Eau de Toilette product, an Eau de Parfum, a cologne, a scent booster, an encapsulated fragrance, a fine fragrance, a men's fine fragrance, a women's fine fragrance, a perfume, a solid perfume, a natural spray product, a perfume spray product, a home care product, a reed diffuser, an all-purpose cleaner, a floor cleaner, a toilet cleaner, a toilet rim block, a bath tissue, liquid air freshener, air freshener spray, a spray dispenser product, an incense stick, a rug deodorizer, a candle, a room deodorizer, a liquid dish detergent, a paste dish detergent, an anti-inflammatory balm, an anti-inflammatory ointment, an anti-inflammatory spray, a disinfectant, a personal care product, a soap, a bar soap, a liquid soap, a bath fragrance, a body wash, a non-aerosol body spray, a body milk, a cleanser, a body cream, a hand sanitizer, a hand wash, a functional product base, a sunscreen lotion, a sunscreen spray, a deodorant, an anti-perspirant, an roll-on product, an aerosol product, a natural spray product, a wax-based deodorant, a glycol type deodorant, a soap type deodorant, a facial lotion, a body lotion, a hand lotion, a miscellaneous lotion, a body powder, a shave cream, a shave gel, a shave butter, a bath soak, a shower gel, an exfoliating scrub, a foot cream, a facial tissue, a cleansing wipe, a talc product, a hair care product, a hair care with ammonia, a shampoo, a hair conditioner, a hair rinse, a hair refresher, a hair fixative or styling aid, a hair bleach, a hair dye or colorant, a fabric care product, a fabric softener, a liquid fabric softener, a fabric softener sheet, a drier sheet, a fabric refresher, an ironing water, a detergent, a laundry detergent, a liquid laundry detergent, a powder laundry detergent, a tablet laundry detergent, a laundry detergent bar, a laundry detergent cream, a hand wash laundry detergent, an insect repellent product, a wildlife scent, and the like. The antioxidant composition can also be added to a cosmetic product, a flavor composition, or a food product.

As indicated above, a fragrance composition or a consumer product can contain an effective amount of the antioxidant composition of this invention that is substantially stable with respect to the fragrance ingredients in an amount sufficient to stabilize the fragrance against oxidative degradation.

The invention is described in greater detail by the below non-limiting examples. Without further elaboration, it is believed that one skilled in the art can, based on the description herein, use the present invention to its fullest extent. All publications cited herein are incorporated by reference in their entirety.

### EXAMPLES 1 and 2

Two antioxidant compositions of this invention, i.e., Compositions 1 and 2, were prepared by mixing the ingredients in Table 1 below.

TABLE 1

| Ingredient, wt % | Composition 1 | Composition 2 |
|---|---|---|
| methyl 3-(3,5-di-tert-butyl-4- | 25% | — |
| hydroxyphenyl) propanoate | | |
| α-tocopherol | — | 25% |
| tris(tetramethylhydroxypiperidinol) | 1.5% | 1.5% |
| citrate | | |
| citric acid | 0.25% | 0.25% |
| benzyl benzoate | 29.3% | — |
| triethyl citrate | 29.3% | — |
| dipropylene glycol | 14.65% | 73.25% |

In Compositions 1 and 2, methyl 3-(3,5-di-tert-butyl-4-hydroxyphenyl) propanoate and α-tocopherol are the primary oxidants, and benzyl benzoate, triethyl citrate and dipropylene glycol are solvents. These two compositions are readily added to a product base such as a fragrance oil, a hydro-alcoholic system, or a consumer product such as an Eau de Toilette, a body spray, or a hand sanitizer. The compositions are in a liquid form and stabilize the fragrance oil, hygro-alcoholic system, and consumer product. The concentration of each ingredient can be adjusted so that the composition thus prepared is compatible with the product base.

### Fragrance 1

Fragrance 1 was prepared by mixing the ingredients listed in Table 2 below. An antioxidant composition was added to Fragrance 1 to assess its antioxidant efficacy.

TABLE 2
Fragrance 1

| Ingredient Name | CAS number | Amount (wt %) |
|---|---|---|
| Decanal | 112-31-2 | 10 |
| Citronellal | 106-23-0 | 10 |
| Iso cyclo citral | 1335-66-6 | 10 |
| Linalool | 78-70-6 | 10 |
| Dihydromyrcenol | 18479-58-8 | 10 |
| Decanol | 112-30-1 | 10 |
| Limonene | 5989-27-5 | 10 |
| Alpha-terpinene | 99-86-5 | 10 |
| Cis-jasmone | 488-10-8 | 10 |
| Menthone | 89-80-5 | 10 |

### Fragrance 2

Fragrance 2 was prepared with 10 wt % of Fragrance 1, 78 wt % ethanol, and 12 wt % water. Fragrance 2 is a hydro-alcoholic formulation based on Fragrance 1.

### EXAMPLES 3-7

Compositions 3-7 were prepared by adding a primary oxidant (α-tocopherol or MDTBHPP), citric acid, and optionally TTMHPC at the level indicated in Table 3 below by weight of Fragrance 1.

Compositions 3 and 4 contains α-tocopherol and citric acid, at the weight ratios of 1000:1 and 4800:1, respectively.

Composition 5 contains α-tocopherol, TTMHPC, and citric acid at the weight ratio of 100:6:1.

Composition 6 contains MDTBHPP and citric acid at the weight ratio of 100:1.

Composition 7 contains MDTBHPP, TTMHPC, and citric acid at the weight ratio of 100:6:1.

Composition 8 contains MDTBHPP, TTMHPC, and citric acid at the weight ratio of 20:10:1.

Ten control compositions were also prepared with the ingredients and their level in Table 3 by weight of Fragrance 1.

TABLE 3

| | α- | | | citric | |
|---|---|---|---|---|---|
| | tocopherol | MDTBHPP | TTMHPC | acid | BHT |
| Composition | wt % | wt % | wt % | wt % | wt % |
| Composition 3 | 0.1 | 0 | 0 | 0.001 | 0 |
| Composition 4 | 0.48 | 0 | 0 | 0.001 | 0 |
| Composition 5 | 0.1 | 0 | 0.006 | 0.001 | 0 |
| Composition 6 | 0 | 0.1 | 0 | 0.001 | 0 |
| Composition 7 | 0 | 0.1 | 0.006 | 0.001 | 0 |
| Composition 8 | 0 | 0.06 | 0.03 | 0.003 | 0 |
| Control-0 | 0 | 0 | 0 | 0 | 0 |
| Control-citric | 0 | 0 | 0 | 0.001 | 0 |
| Control-TT | 0 | 0 | 0.006 | 0 | 0 |
| Control-CT | 0 | 0 | 0.006 | 0.001 | 0 |
| Control-BHT-0.1 | 0 | 0 | 0 | 0 | 0.1 |
| Control-BHT-0.25 | 0 | 0 | 0 | 0 | 0.25 |
| Control-BHT-0.5 | 0 | 0 | 0 | 0 | 0.5 |
| Control-TOCO-0.1 | 0.1 | 0 | 0 | 0 | 0 |
| Control-TOCO-0.48 | 0.48 | 0 | 0 | 0 | 0 |
| Control-M-1 | 0 | 0.1 | 0 | 0 | 0 |

### Efficacy of Proposed Antioxidant Formulations on a Mix of Oxidation Sensitive Raw Materials in Oil Media.

Compositions 3-7 and the ten control compositions were evaluated for their antioxidant efficacy using a RapidOxy instrument, which allows an accelerated oxidation process under elevated temperature and excessive oxygen.

The compositions and controls (5 mL each) were tested at a temperature of 80° C. and a pressure of 300 kPa, with 50% pressure drop below Pₘₐₓ. The time to consumer 0.25 mol/L of oxygen (O₂) was recorded in hours (h) for each composition. A longer time indicates higher antioxidant efficacy. The results are shown in Table 4 below.

TABLE 4

| | Time to consume 0.25 |
|---|---|
| Composition | mol/L of O₂ (h) |
| Control-0 | 2.96 |
| Control-citric | 3 |
| Control-TT | 8 |
| Control-CT | 8.3 |
| Control-BHT-0.1 | 15.4 |
| Control-BHT-0.25 | 18.4 |
| Control-BHT-0.5 | 19.5 |
| Control-TOCO-0.1 | 4.3 |
| Control-TOCO-0.48 | 11 |
| Control-M-1 | 9.3 |
| Control-M-2 | 13.1 |
| Composition-3 | 12.1 |
| Composition 4 | 12.1 |
| Composition 5 | 16.2 |
| Composition 6 | 11.6 |
| Composition 7 | 20.7 |

Surprisingly, Composition 3 took 12.1 hours to consume 0.25 mol/L of oxygen, a much longer time than the α-tocopherol control (4.3 hours) and the citric acid control (3 hours). Theoretically, the time required to consumer 0.25 mol/L of oxygen by α-tocopherol and citric acid would be 4.3 hours without any synergy effect.

Composition 5 took 16.2 hours to consume 0.25 mol/L of oxygen. As shown in Table 3 above, Composition 5 contained 0.1% of α-tocopherol, 0.006% of TTMHPC, and 0.001% of citric acid. Accumulatively, it would take 9.4 hours for these three ingredients to consume 0.25 mol/L of oxygen, calculated as follows: time of Control-citric+time of Control-TT+time of Control-TOCO-0.1-2×time of Control 0.

Composition 6 took 11.6 hours to consume 0.25 mol/L of oxygen, a much longer time than that of individual components if no synergy exists (9.3 hours).

Composition 7 took 20.7 hours to consume 0.25 mol/L of oxygen. Without synergy effect, it would only take 14.4 hours if MDTBHPP, TTMHPC, and citric acid were calculated accumulatively.

Efficacy of Antioxidant Compositions of this Invention on Commercial Fragrance Products

Six hydroalcoholic fragrance products, i.e., Fragrances I-VI, were used to evaluate efficacy of three antioxidant compositions of this invention (Compositions 5 and 7-8). Fragrances I-VI each contain a different fragrance oil dissolved in ethanol and water with the concentrations indicated below in Table 5.

TABLE 5
Fragrances I-VI

| Fragrance | | | |
|---|---|---|---|
| Products | Fragrance oil (%) | Ethanol (%) | Water (%) |
| Fragrance I | 25 | 67 | 8 |
| Fragrance II | 20 | 79 | 1 |
| Fragrance III | 25 | 67 | 8 |
| Fragrance IV | 10 | 78 | 12 |
| Fragrance V | 12 | 78 | 10 |
| Fragrance VI | 11 | 78 | 11 |

Efficacy of Compositions 5, 7-8 and control compositions is assessed against the fragrance products by adding the compositions to the fragrance oils or directly to the fragrance products at the levels indicated in Table 3 above.

Each fragrance product was added an antioxidant composition or BHT to obtain a protected fragrance product, which was stored for four weeks at 50° C. Control-0 was the fragrance product without adding any antioxidant and was also stored at 50° C. The original fragrance product was stored for four weeks at 5° C. as the reference fragrance. A trained panel assessed each sample through organoleptic evaluations (odor and color).

Odor is evaluated through two criteria, i.e., intensity and quality. After the storage, the differences between each fragrance product and the reference fragrance were recorded in Table 7 below. As shown in this table, the first digit characterizes a change in fragrance intensity, on a scale from 0 to 5, with a score of 0 indicating no change, and a score of 5 indicating a huge change. The second digit characterizes a change in fragrance quality, also on a scale from 0 to 5. The color of each fragrance product was also evaluated and compared. Table 7 includes the color change on a scale of A to F between a protected fragrance product and Control-0 after the storage. A score of A indicates no change and a score of F indicates a huge change. See Table 6 below for the evaluation scores in odor and color.

TABLE 6
Odor and color scores

| Fragrance intensity/ | Fragrance | Degree of | |
|---|---|---|---|
| quality change | color change | change | Acceptability |
| 0 | A | No change | Yes |
| 1 | B | Not significant change | |
| 2 | C | Slight change | |
| 3 | D | Significant change | Limited |
| | | | acceptance |
| 4 | E | Important change | No |
| 5 | F | Huge change | |

TABLE 7
Odor and color evaluation

| | | Control- | | |
|---|---|---|---|---|
| | Control- | BHT- | | |
| | 0 | 0.25 | Composition 5 | Composition 7 |
| Fragrance I | 03E | 03D | — | 02D |
| Fragrance II | 02D | 02D | 01D | 01C |
| Fragrance III | 02E | 01D | — | 01D |
| Fragrance IV | 04A | 02A | 03B | 02A |
| Fragrance V | 02C | 01D | 01D | 01B |
| Fragrance VI | 03E | 02E | 02E | 02E |

Results in Table 7 show that Compositions 5 and 7 of this invention protected fragrance products from discoloration and quality changes.

In addition, Composition 8 of this invention was evaluated in Fragrances I, II, and VI, together with Control-0 and Control-BHT-0.05 (0.05% by weight of the fragrance product).

Efficacy of Composition 8 was assessed by GC-MS for the recovery of fragrance oil after stored for two weeks at 60° C. as compared to the reference fragrance stored at 5° C. ΔE_{CMC} was also calculated for Composition 8 after stored at 60° C. for two weeks using the reference fragrance stored at 5° C. as the base. ΔE_{CMC} is calculated using Hunter L, a, b color scales based on the Opponent-Color Theory. This theory assumes that the receptors in the human eye perceive color as the following pairs of opposites. The L value for each scale therefore indicates the level of light or dark, the a value redness or greenness, and the b value yellowness or blueness. All three values are required to completely describe an object's color. The delta values (ΔL, Δa, and Δb) indicate how much a composition and a reference differ from one another in L, a, and b. The total color difference, ΔE_{CMC} is thus calculated as a single value that considers the differences between the L, a, and b of the composition and the reference. A high ΔE_{CMC} value indicates a great color difference.

Table 8 below shows the GC-MS recovery and ΔE_{CMC} of Composition 8 as compared to Control-0 and Control-BHT-0.05 in three fragrances.

TABLE 8
Recovery and ΔE_{CMC} after storage at 60° C. for two weeks

| | GC-MS recovery (%) | | | ΔE_{CMC} | | |
|---|---|---|---|---|---|---|
| | | Control- | | | Control- | |
| | Control- | BHT- | Composition | Control- | BHT- | Composition |
| Composition | 0 | 0.05 | 8 | 0 | 0.05 | 8 |
| Fragrance I | 72 | 80 | 78 | 4.0 | 2.4 | 2.9 |
| Fragrance II | 84 | 90 | 95 | 0.3 | 0.3 | 0.3 |
| Fragrance VI | 90 | 92 | 94 | 3.4 | 3.3 | 1.2 |

Table 8 shows that Composition 8 effectively protect fragrance products with high recovery and low color change.

Efficacy of Antioxidant Compositions of this Invention for Fragrances in Shampoo and Conditioner Products

Compositions 5 and 7 were added to Fragrance VII separately to obtain Fragrance VII-5 and VII-7, which were added to a shampoo composition and a hair conditioner composition to evaluate changes in product odor and color using the scales described above.

Each fragranced shampoo or hair conditioner product containing an antioxidant composition or control was stored at 45° C. for eight weeks. A reference product was stored at 5° C., which contained the fragrance but not any antioxidant composition. The organoleptic evaluation (odor and color) results are shown in Table 9 below.

TABLE 9
Antioxidant compositions in shampoo and hair conditioner products

| | Control- | Control- | Composition | Composition |
|---|---|---|---|---|
| | 0 | BHT-0.4 | 5 | 7 |
| Shampoo | 03C | 02B | 03C | 02B |
| Conditioner | 03C | 02C | 02C | 01B |

Results in Table 9 show that Compositions 5 and 7 of this invention effectively protect the fragrance in the shampoo and hair conditioner products.

1. An antioxidant composition comprising:
(a) a primary antioxidant, and
(b) an organic acid.

2. The antioxidant composition of claim 1, wherein the primary antioxidant is α-tocopherol, methyl 3-(3.5-di-tert-butyl-4-hydroxyphenyl) propanoate, or a combination thereof.

3. The antioxidant composition of claim 1, wherein the organic acid is selected from the group consisting of citric acid, oxalic acid, malic acid, and combinations thereof.

4. The antioxidant composition of claim 1 further comprising tris(tetramethylhydroxypiperidinol) citrate.

5. The antioxidant composition of claim 1 further comprising a solvent that is polar solvent, a non-polar solvent, or a combination thereof.

6. The antioxidant composition of claim 5, wherein the solvent is benzyl benzoate, triethyl citrate, dipropylene glycol, or a combination thereof.

7. The antioxidant composition of claim 1, wherein the weight ratio of the primary antioxidant and the organic acid is 500:1 to 1:50, preferably 300:1 to 1:20, and more preferably 200:1 to 1:10.

8. The antioxidant composition of claim 4, wherein the weight ratio of the primary antioxidant and tris(tetramethylhydroxypiperidinol) citrate is 500:1 to 1:50, preferably 300:1 to 1:20, and more preferably 200:1 to 1:10.

9. A fragrance composition comprising a fragrance and the antioxidant composition of claim 1.

10. A consumer product comprising a consumer product base and the antioxidant composition of claim 1.

11. The fragrance composition of claim 9, wherein the primary antioxidant is present at a level of 0.001 wt % to 1 wt %, preferably 0.01 wt % to 0.3 wt %, and more preferably 0.01 wt % to 0.2 wt %; the organic acid is present at a level of 0.00001 wt % to 2 wt %, preferably 0.0001 wt % to 1 wt %, and more preferably 0.0001 wt % to 0.5 wt %; and tris(tetramethylhydroxypiperidinol) citrate is present at a level of 0 to 1 wt % (w/w), preferably 0.00001 wt % to 0.5 wt %, and more preferably 0.0001 wt % to 0.1 wt %.

12. The consumer product composition of claim 10, wherein the primary antioxidant is present at a level of 0.001 wt % to 1 wt %, preferably 0.01 wt % to 0.3 wt %, and more preferably 0.01 wt % to 0.2 wt %; the organic acid is present at a level of 0.00001 wt % to 2 wt %, preferably 0.0001 wt % to 1 wt %, and more preferably 0.0001 wt % to 0.5 wt %; and tris(tetramethylhydroxypiperidinol) citrate is present at a level of 0 to 1 wt % (w/w), preferably 0.00001 wt % to 0.5 wt %, and more preferably 0.0001 wt % to 0.1 wt %.
